(19) United States
(12) Patent Application Publication
(10) Pub. No.: US 2004/0133935 A1
(43) Pub. Date: Jul. 8, 2004

(54) Preparation method of donor cell for nuclear transfer using electro-stimulation and membrane antigen marker and production method of clone animal

(75) Inventors: Tomohiro Kono, Tokyo-city (JP); Oh-Yong Kwon, Seoul (KR); Yukiko Ono, Kanagawa-ken (JP)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Macrogen Inc., Seoul (KR)

(21) Appl. No.: 10/690,049

(22) Filed: Oct. 21, 2003

(30) Foreign Application Priority Data
Oct. 21, 2002 (KR) 2002-0064202

(51) Int. Cl. C12N 15/02

(52) U.S. Cl. 800/21; 435/450

(57) ABSTRACT

The present invention relates to a method for preparing a donor cell for nuclear transfer using electro-stimulation and a membrane antigen marker, and a method for producing clone animals using the same. Particularly, the present invention provides a method for improving productivity of clone animals and knock-out animals using electro-stimulated donor cells and/or undifferentiated donor cells.

### BACKGROUND OF THE INVENTION

(a) Field of the Invention

The present invention relates to a method for preparing a donor cell for nuclear transfer using electro-stimulation and a membrane antigen marker, and a method for producing a clone animal, and more particularly to a method for improving productivity of knock out animals or clone animals by selecting only undifferentiated donor cells and conducting nuclear transfer.

(b) Description of the Related Art

An embryonic stem cell, which is separated from the inner cell mass of a blastocyst that is the initial stage of a fertilized egg grown for about 5 days, has totipotency such that it is possible for it to differentiate into all cells. Since the embryonic stem cell can be artificially injected into a gamete, it can be differentiated into gametes. Therefore, studies on separating embryonic stem cells have long been undertaken due to their usefulness, and the production of knock out animals for elucidating the function of a gene at an individual level using an embryonic stem cell has been accomplished.

A knock out mouse, which is a transformed mouse from which a specific gene is removed, is produced by introducing a vector capable of removing a specific gene by homologous recombination into an embryonic stem cell, selecting the transformed embryonic stem cell, injecting the selected transformed embryonic stem cell into a fertilized egg of a blastocyst to implant in a surrogate mother, and then hybridizing the delivered transformed mice.

However, the production of a knock out mouse requires a lot of effort, time, and money, and it is likely that the knock out mouse will become sterile.

In addition, knock out animals can be produced by nuclear transfer. A nuclear transfer is the transfer of donor nuclear cells into an enucleated recipient oocyte, and it is conducted by cell fusion and introcytoplasmic cell injection (ICCI). The cell fusion includes exposure to chemicals, injection into an inactivated Sendai virus, electrophoresis, and intracellular nuclear injection through intracytoplasmic sperm injection (ICSI). However, such a nuclear transfer has various problems such as low fetal production rate, abnormal fetal delivery, etc.

### SUMMARY OF THE INVENTION

The present invention is made in order to solve the problems of the prior art, and it is an object of the present invention to provide a method for producing a clone animal by nuclear transfer between embryonic stem cells and a recipient oocyte, which can improve productivity of clone animals.

It is another object of the present invention to provide a method for pre-treating an embryonic stem cell, which can improve productivity of clone animals.

It is another object of the present invention to provide a method for stably producing a knock out animal.

In order to achieve these objects, the present invention provides a method for preparing a donor cell for nuclear transfer comprising the steps of (a) applying electrical stimulation on donor cells, and (b) conducting synchronous culture of the electrically stimulated donor cells to a metaphase stage of the cell division cycle.

The present invention also provides a method for preparing a donor cell for nuclear transfer comprising the steps of (a) reacting donor cells with an antibody against a membrane antigen marker that is specifically expressed in undifferentiated cells, (b) identifying the antigen-antibody reaction in the reacted donor cells to select undifferentiated donor cells, and (c) conducting synchronous culture of the undifferentiated donor cells to a metaphase stage of the cell division cycle.

The present invention also provides a nuclear transfer method comprising the steps of (a) preparing a donor cell by the above preparation method of a donor cell for nuclear transfer, and (b) conducting nuclear transfer of the prepared donor cell into a recipient cell from which chromosomes are removed.

The present invention also provides a method for producing an animal comprising the steps of (a) reconstituting an animal embryo by the above nuclear transfer method, and (b) generating an animal from the embryo.

The present invention also provides a donor cell for nuclear transfer prepared by the above preparation method of a donor cell for nuclear transfer.

The present invention also provides a reconstituted embryo prepared by the above nuclear transfer method.

The present invention also provides an animal produced by the above animal production method.

### BRIEF DESCRIPTION OF THE DRAWINGS

FIG. 1 shows an antibody that can select a membrane antigen marker existing on a membrane of a donor cell, and an antigen-antibody complex.

FIG. 2 show a method for selecting only undifferentiated donor cells after reacting a donor cell with an antibody marked with a magnetic bead.

### DETAILED DESCRIPTION OF THE PREFERRED EMBODIMENTS

The present inventors developed a method for preparing a donor cell that can improve productivity of animals, by applying electrical stimulation to a donor cell for nuclear transfer and/or selecting undifferentiated donor cells.

A donor cell is a cell from which the nucleus is transferred to a recipient cell, and includes common somatic cells and gametes. Preferably, it is an embryonic stem cell or stem cell, and more preferably an embryonic stem cell or stem cell that is synchronously cultured to the metaphase of the cell division cycle. The donor cell can be a wild-type animal cell, or it can be a wild-type animal cell into which a specific gene is inserted or one that has been operated upon to modify its chromosomes by genetic engineering means. The genetic engineering means include gene transfer or knock out, which can be easily conducted by an ordinarily skilled person.

A recipient cell is a cell from which the original nucleus is removed through enucleation, and to which the nucleus is transferred from a donor cell. Any common cell can be a recipient cell, but in the present invention, a cell of which an embryo is reconstituted by nuclear transfer to have a potential of generating an animal is preferable, a representative example being an oocyte, and more preferably an oocyte at the metaphase of meiosis II.

As the donor cell and recipient cell, animal cells are preferable, and concrete examples include those derived from mammals including mice, rats, goats, sheep, pigs, cows, monkeys, and humans. The donor cell and recipient cell can be cultured or pre-treated according to a common method used in nuclear transfer.

The method for preparing a donor cell of the present invention comprises the steps of applying electrical stimulation to donor cells and conducting synchronous culture of the electrically stimulated donor cells to the metaphase of cell division. The method can further comprise the step of reacting the electrically stimulated donor cells with an antigen against a membrane antigen marker that is specific to undifferentiated cells to select undifferentiated donor cells, and the selected undifferentiated donor cells can be synchronously cultured to the metaphase of meiosis.

In the present invention, the electrical stimulation can be conducted at a voltage of 200 to 350 V, a condenser value of 900 to 1000 uF, and a resistance value of infinity, for 10 to 30 msec, and it is preferably conducted at a voltage of 266 to 300 V, a condenser value of 975 uF, and a resistance value of infinity, for 17.8 to 21.6 msec. If the voltage, condenser value, and electrical stimulation time do not fall within the above ranges, the animal production rate by nuclear transfer can be lowered.

Another method for preparing a donor cell for nuclear transfer of the present invention comprises the steps of (a) reacting donor cells with an antibody against a membrane antigen marker that is specifically expressed in undifferentiated cells, (B) identifying an antigen-antibody reaction in the reacted donor cells to select undifferentiated donor cells, and (c) conducting synchronous culture of the undifferentiated donor cells to the metaphase of cell division. The method can further comprise the step of applying electrical stimulation to donor cells before the step of (a).

The membrane antigen marker includes all kinds of proteins that are specifically expressed in undifferentiated cells and located in a cell membrane, and is preferably selected from the group consisting of SSEA-1 (stage-specific embryonic antigen-1), CD117 (c-kit), sca-1 (Stem Cell Antigen), and CD31 (PECAM-1, Platelet Endothelial Cell Anhesion Molecule-1). Particularly, SSEA-1 begins to be expressed at the 8-cell stage and continues until undifferentiated states of morula and inner cell mass occur, and stops being expressed when the cell is differentiated.

In the step of selecting undifferentiated donor cells, it is determined whether or not an antigen-antibody complex between an antibody and a membrane antigen marker located on a donor cell membrane forms, to separate undifferentiated donor cells, and the formation of an antigen-antibody complex can be identified by a common immunoassay. The antibody is a monoclonal antibody or a polyclonal antibody against a membrane antigen marker.

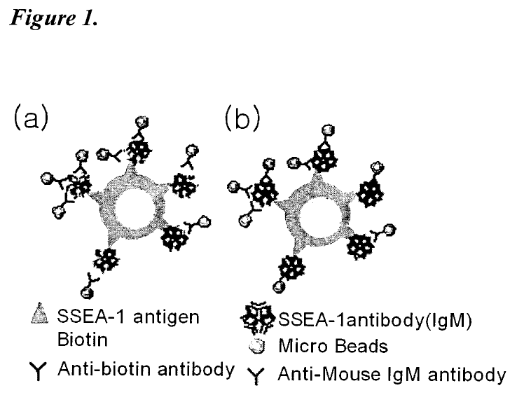

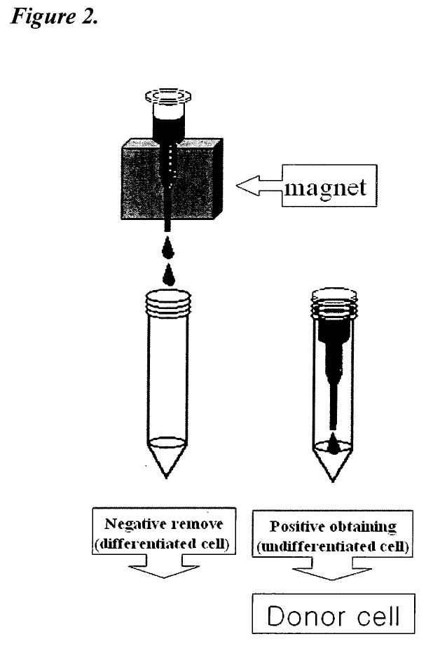

In the present invention, an antigen-antibody complex was analyzed using a magnetic field (FIGS. 1 and 2). This method is to react a donor cell with an antibody marked with micro magnetic beads, and separate a donor cell in which an antigen-antibody complex forms, or to separate a donor cell in which an antigen-antibody complex forms using a secondary antigen marked with micro magnetic beads after using a primary antibody.

More particularly, as shown in FIG. 1, biotin is joined to a membrane antigen marker antibody, which is reacted with a membrane antigen marker, and then an anti-biotin antibody to which a magnetic bead is bound can be secondarily bound thereto (FIG. 1a). Alternatively, after reacting a membrane antigen marker antibody with a membrane antigen marker, an anti-IgM antibody can be secondarily bound (FIG. 1b). The thus-formed antigen-antibody complex can select an undifferentiated donor cell which expresses a membrane antigen marker using a magnetic bead included in the complex.

The donor cell prepared according to the method of the present invention can transfer a nucleus to a recipient by cell fusion or intracytoplasmic cell injection, preferably by cell fusion.

In addition, the present invention provides a reconstituted embryo prepared by a method comprising the steps of (a) preparing a donor cell by the method of preparation of the present invention; and (b) conducting nuclear transfer of the donor cell into a recipient cell from which chromosomes are removed.

The step (b) can be conducted by a known method, and the present invention uses, for example, nocodazole treatment.

The step (c) can be conducted by cell fusion using a Sendai virus, electro-stimulation, or intracytoplasmic cell injection, which can be easily conducted by an ordinarily skilled person.

In addition, the present invention provides a method for producing an animal comprising the steps of reconstituting an animal embryo by the above nuclear transfer method, and generating an animal from the embryo. The step of generating an animal can be conducted by implanting the reconstituted embryo on the uterine wall of a fertile animal for gestation, and making it deliver.

The preparation method of a donor cell for nuclear transfer of the present invention can efficiently produce clone animals by improving productivity.

The present invention will be explained in more detail with reference to the following Examples. However, these are only to illustrate the present invention and the present invention is not limited to them.

### EXAMPLES

### Example 1

### Nuclear Transfer Using an Electrically Stimulated Donor Cell

1-1. Culture and Electro-Stimulation of Embryonic Stem Cell.

Lyophilized embryonic stem cells (6×10⁵ cells) were thawed, and they were seeded on three sheets of 60 mm diameter dishes containing feeder cells obtained from fetal mouse fibroblast. As a culture liquid, knockout-DMEM (Gibco BRL, Grand Island, N.Y.) comprising 15% (v/v) FBS (Fetal Bovine Serum, Gibco), 103 u/ml leukemia inhibitory factor (Wako Pure Chemical Industries, Osaka), 2 mM L-glutamine (Gibco), 1% (v/v) non-essential amino acid solution (Gibco), and 5.5×10⁻⁵ M 2-mercaptoethanol (Wako) was used.

The embryonic stem cell was cultured in a culture medium under conditions of 5% CO₂, 5% O₂, and 90% N₂ for 2 days, exchanging the culture liquid once a day. When the embryonic stem cell became confluent, it was treated with a 0.05-0.53 M trypsin EDTA solution for 5 minutes to extricate it from the cell, and it was diluted with 0.4 ml HBS buffer solution and electrical stimulation was applied.

The electrical stimulation was applied under conditions of a voltage of 266 V, a condenser value of 975 uF, and a resistance value of infinity, for 20 msec. The embryonic stem cell was placed on ice for 10 minutes immediately after electrical stimulation, and then seeded on a feeder cell in a dish with a diameter of 60 mm to culture.

1-2. Synchronous Culture of Embryonic Stem Cell to Metaphase Stage of Cell Division

The embryo stem cells were initially cultured with feeder cells after applying electrical stimulation, and 3 days after initial culture they were cultured in a culture liquid to which nocodazole (0.4 ug/ml) was added, for 2-4 hours. Cells in the metaphase stage lose their adhesive property and rise up in the culture liquid. Therefore, the upper part of the culture liquid was recovered and centrifuged at 1000 rpm for 5 minutes to obtain embryonic stem cells of the metaphase stage.

1-3. Nuclear Transfer and Artificial Culture

To a female B6CBF1(C57BL/6×CBA) mouse, PMSG (pregnant mare serum gonadotropin) and hCG (human chorionic gonadotropin) were respectively administrated with an interval of 48 hours therebetween, and 14 hours after hCG administration a non-fertilized egg was recovered from a swollen part of the uterine tube. A small protruding part of cytoplasm of the non-fertilized egg or a transparent part nearest to a more transparent part than the cytoplasm (a part where chromosomes existence is observed) was incised with a micro-operator. Then, on an M2 liquid phase containing cytochalasin B (5 ug/ml), a glass tube for micro-operation was inserted into a crevice of the incised transparent part, and chromosomes and some cytoplasm were suctioned to remove them. The non-fertilized egg from which chromosomes were removed was fused with an embryo stem cell of the metaphase stage in a Sendai virus (Kono, T., Kwon, O Y., & Nakahara, T. (1991) Journal of Reproduction Fertility and Kono, T., Sotomaru, Y., Aono, F., Takahashi, T., Ogiwara, I., Sekzawa, F., Arai, T., & Nakahara, T. (1994) Theriogenology 41, 1463-1471). Nuclear transfer was conducted on an M2 culture liquid containing cytochalasin B (5 ug/ml, Sigma) and nocodazole (0.4 ug/ml).

The fused cell was cultured for 2 hours in a CZB culture liquid, and 6 hours in 10 mM strontium solution. An oocyte having one polar body and one pronucleus was transferred to a CZB culture liquid, and cultured in a culture medium under conditions of 5% CO₂, 5% O₂, and 90% N₂ for 4 days. The oocyte of the blastocyst stage was hybridized with a sterilized male, and after 2.5 days it was transferred onto the uterine wall of a surrogate mother, and at the 19.5th day of pregnancy, it was determined through an operation whether or not a fetus existed.

### Example 2

### Nuclear Transfer After Selecting Undifferentiated Cells Using a Membrane Antigen Marker

An experiment was conducted by the same method as in Example 1, except that electrical stimulation was not applied, and the following experiments were further conducted before steps 1-3 of Example 1.

Selection of Undifferentiated Cells Using a Membrane Antigen Marker

In order to allow separation of positive (+) cells for a membrane surface antigen and negative cells (−) with a magnetic column (Milteniyi Biotec), embryonic stem cells were magnetically marked using micro beads.

Metaphase stage embryonic stem cells were reacted with 7 ug/100 ul of an antibody (anti mouse SSEA-1) at 37° C. for 1 hour. Dead cells were removed (Dead cell removal kit: Milteniyi Biotec #130-090-101), and a micro-bead-marked secondary antibody (Rat anti-mouse IgM micro beads: Milteniyi Biotec #130-047-302) was introduced to react at 6 to 12° C. for 15 minutes. Then, in the magnetic field of the reacted embryonic stem cells, a separation column was installed to separate embryonic stem cells in which an antigen-antibody complex formed (FIG. 1).

### Example 3

### Nuclear Transfer After Selection of Undifferentiated Cells Using Electro-Stimulation and a Membrane Antigen Marker.

The experiment was conducted by the same method as in Example 1, except that undifferentiated cell selection was conducted using a membrane antigen marker as described in Example 2 before steps 1-3 of Example 1 to conduct nuclear transfer using an undifferentiated cell as a donor cell, and after implanting on the uterine wall of a surrogate mother, productivity was confirmed.

### Comparative Example 1

The experiment was conducted by the same method as in Example 1, except that electrical stimulation was not applied to the embryonic stem cell.

### Comparative Example 2

Nuclear transfer was conducted by the same method as in Example 3, except that a differentiated embryonic stem cell that was judged to be negative in undifferentiated cell selection using a membrane antigen was used as a donor cell for nuclear transfer.

Experiment

(1) Examination of Productivity According to Electro-Stimulation of Embryonic Stem Cell

No. of implantation sites, implantation rate, and no. of live pups according to Example 1 and Comparative Example 1 were measured, and results are shown in Table 1.

TABLE 1

| | No. of | | No. | | |
|---|---|---|---|---|---|
| | nuclear | | pregnants/ | No. of | |
| | transferred | No. of | no. of | implan- | No. of |
| | embryos | blastocysts | recipients | tation | live |
| | cultured | transferred | (%) | sites (%) | pups(%) |
| Comparative | 320 | 148(46.3) | 5/12(41.7) | 24(16.2) | 0(0) |
| Example 1 | | | | | |
| Example 1 | 245 | 110(44.9) | 6/9(66.7) | 57(51.9) | 3(2.7) |

As can be seen from the Table 1, Example 1 wherein electrical stimulation was applied shows higher implantation and productivity rates than Comparative Example 1.

(2) Examination of Productivity According to Differentiation of Embryonic Stem Cell

In order to examine productivity according to differentiation of an embryonic stem cell used as a donor cell, no. of implantation sites, implantation rate, and no. of live pups of the reconstituted eggs according to Example 3 and Comparative Example 2 were measured and results shown in the following Table 2.

TABLE 2

| | No. of nuclear | | | |
|---|---|---|---|---|
| | transferred | No. of | No. of | |
| | embryos | blastocysts | implantation | No. of live |
| | cultured | transferred (%) | sites (%) | pups (%) |
| Comparative | 55 | 37(67) | 13(24) | 0(0) |
| Example 2 | | | | |
| Example 3 | 70 | 51(73) | 39(56) | 3(5.9) |

As seen from Table 2, Example 3 using an undifferentiated embryonic stem cell as a donor cell shows a high blastocyst stage appearance rate, implantation rate, and productivity, while Comparative Example 2 using a differentiated embryonic stem cell shows a lower success rate compared to Example 3.

The method for preparing a donor cell for nuclear transfer of the present invention can prepare a donor cell capable of improving animal productivity in an easy and simple manner, thereby efficiently producing a clone animal or knock out animal.

What is claimed is:

1. A method for preparing a donor cell for nuclear transfer, comprising the steps of:
(a) applying electrical stimulation to donor cells; and
(b) conducting synchronous culture of the electrically stimulated donor cells to a metaphase stage.

2. The method for preparing a donor cell for nuclear transfer according to claim 1, wherein the electrical stimulation is conducted at a voltage of 200 to 350 V, a condenser value of 900 to 1000 uF, and a resistance value of infinity, for 10 to 30 msec.

3. The method for preparing a donor cell for nuclear transfer according to claim 1, wherein the method further comprises the step of reacting the electrically stimulated donor cells of the step (a) with an antibody against a membrane antigen marker that is specific to undifferentiated cells to select undifferentiated donor cells, before the step of (b).

4. The method for preparing a donor cell for nuclear transfer according to claim 1, wherein the donor cell is an embryo stem cell.

5. The method for preparing a donor cell for nuclear transfer according to claim 1, wherein chromosomes of the donor cell are modified by genetic engineering means.

6. The method for preparing a donor cell for nuclear transfer according to claim 1, wherein the nuclear transfer is cell fusion.

7. A method for preparing a donor cell for nuclear transfer, comprising the steps of:
(a) reacting donor cells with an antibody against a membrane antigen marker that is specifically expressed in undifferentiated cells;
(b) identifying an antigen-antibody reaction in the reacted donor cells to select undifferentiated donor cells; and
(c) conducting synchronous culture of the undifferentiated donor cells to a metaphase stage.

8. The method for preparing a donor cell for nuclear transfer according to claim 7, wherein the membrane antigen marker that is specifically expressed in undifferentiated cells is selected from the group consisting of SSEA-1 (stage-specific embryonic antigen-1), CD117(c-kit), sca-1 (Stem Cell Antigen), and CD31 (PECAM-1, Platelet Endothelial Cell Anhesion Molecule-1).

9. The method for preparing a donor cell for nuclear transfer according to claim 7, wherein the donor cell is an embryonic stem cell.

10. The method for preparing a donor cell for nuclear transfer according to claim 7, wherein chromosomes of the donor cell are further modified by genetic engineering means.

11. The method for preparing a donor cell for nuclear transfer according to claim 7, wherein the nuclear transfer is cell fusion.

12. A method of nuclear transfer comprising the steps of:
(a) preparing a donor cell by the method of claim 1; and
(b) conducting nuclear transfer of the prepared donor cell into a recipient cell from which chromosomes are removed.

13. A method of nuclear transfer comprising the steps of:
(a) preparing a donor cell by the method of claim 7; and
(b) conducting nuclear transfer of the prepared donor cell into a recipient cell from which chromosomes are removed

14. A method for producing an animal comprising the steps of:
(a) reconstituting an animal embryo by the method of claim 12; and
(b) generating an animal from the reconstituted embryo.

15. A method for producing an animal comprising the steps of:
(a) reconstituting an animal embryo by the method of claim 13; and
(b) generating an animal from the reconstituted embryo.

16. A donor cell for nuclear transfer prepared by the method of claim 1.

17. A donor cell for nuclear transfer prepared by the method of claim 7.

18. A reconstituted embryo prepared by the nuclear transfer method of claim 12.

19. A reconstituted embryo prepared by the nuclear transfer method of claim 13.

20. An animal produced by the method of claim 14.

21. An animal produced by the method of claim 15.
